(19) United States
(12) Patent Application Publication
(10) Pub. No.: US 2022/0249209 A1
(43) Pub. Date: Aug. 11, 2022

(54) ORAL SCANNER

(71) Applicant: Qisda Corporation, Taoyuan City (TW)

(72) Inventors: Chien-Hung LIN, Taoyuan City (TW); Po-Fu WU, Taoyuan City (TW); Jun-Ming SHEN, Taoyuan City (TW); Szu-Fan CHEN, Taoyuan City (TW); Yi-Ling LO, Taoyuan City (TW)

(73) Assignee: Qisda Corporation, Taoyuan City (TW)

(21) Appl. No.: 17/582,106

(22) Filed: Jan. 24, 2022

(30) Foreign Application Priority Data
Feb. 8, 2021 (CN) 202110184344.2

(51) Int. Cl. A61C 9/00; A61B 1/00; A61B 1/24; G02B 26/08

(52) U.S. Cl. CPC A61C 9/006; G02B 26/0833; A61B 1/24; A61B 1/00172

(57) ABSTRACT

An oral scanner including a heating element, a reflecting element and a temperature difference generating element is provided. The temperature difference generating element has a high temperature end and a low temperature end. The high temperature end is connected to the reflecting element to heat the reflecting element, and the low temperature end is connected to the heating element to cool the heating element.

This application claims the benefit of People's Republic of China application Serial No. 202110184344.2, filed on Feb. 8, 2021, the subject matter of which is incorporated herein by reference.

### BACKGROUND OF THE INVENTION

### Field of the Invention

The invention relates in general to a scanner, and more particularly to an oral scanner.

### Description of the Related Art

When oral scanner operates at an oral cavity, due to the temperature difference between the oral cavity and the interior of the oral scanner, the reflecting mirror of the oral scanner will be atomized. Since some electronic elements inside the oral scanner generate high temperature, the generated heat needs to be dissipated. Therefore, it has become a prominent task for the technicians of the technology field to improve the atomization of the optical lens of the oral scanner and/or resolve the heat dissipation problem of the electronic elements.

### SUMMARY OF THE INVENTION

The invention is directed to an oral scanner capable of resolving the problems encountered in the prior art.

According to one embodiment of the present invention, an oral scanner is provided. The oral scanner includes a heating element, a reflecting element and a temperature difference generating element. The temperature difference generating element has a high temperature end and a low temperature end. The high temperature end is connected to the reflecting element to heat the reflecting element, and the low temperature end is connected to the heating element to cool the heating element.

According to another embodiment of the present invention, an oral scanner is provided. The oral scanner includes a heating element and a temperature difference generating element. The temperature difference generating element has a high temperature end and a low temperature end. The low temperature end is connected to the heating element to cool the heating element.

According to an alternate embodiment of the present invention, an oral scanner is provided. The oral scanner includes a reflecting element and a temperature difference generating element. The temperature difference generating element has a high temperature end and a low temperature end. The high temperature end is connected to the reflecting element to heat the reflecting element.

The above and other aspects of the invention will become better understood with regard to the following detailed description of the preferred but non-limiting embodiment (s). The following description is made with reference to the accompanying drawings.

### BRIEF DESCRIPTION OF THE DRAWINGS

FIG. 1 is a schematic diagram of an oral scanner according to an embodiment of the present invention.

FIG. 2 is a schematic diagram of an oral scanner according to another embodiment of the present invention.

### DETAILED DESCRIPTION OF THE INVENTION

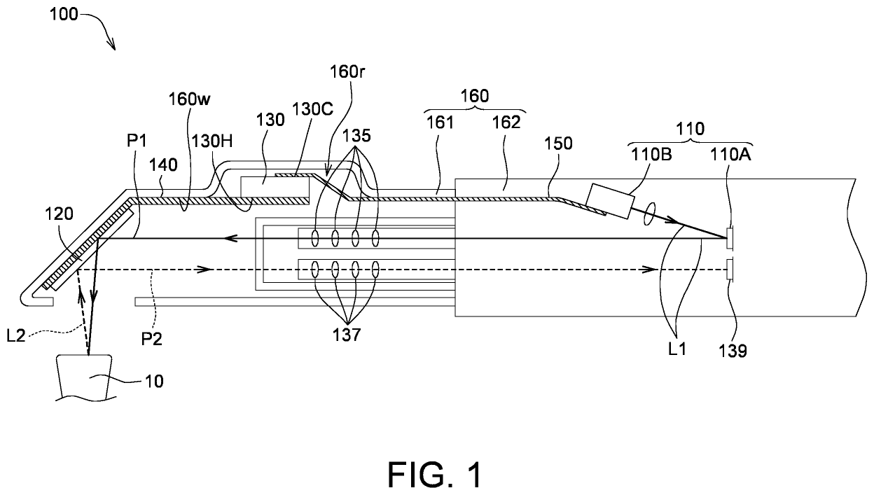

Referring to FIG. 1, is a schematic diagram of an oral scanner 100 according to an embodiment of the present invention is shown. The oral scanner 100 includes a heating element 110, a reflecting element 120, a temperature difference generating element 130, a projection imaging module 135, an image optical module 137, an image sensing element 139, a first heat conductive element 140, a second heat conductive element 150 and a casing 160. The heating element 110, the reflecting element 120, the temperature difference generating element 130, the projection imaging module 135, the image optical module 137, the image sensing element 139, first heat conductive element 140 and second heat conductive element 150 are disposed in the casing 160.

The temperature difference generating element 130 has a high temperature end 130H and a low temperature end 130C. The high temperature end 130H is connected to the reflecting element 120 to heat the reflecting element 120, and the low temperature end 130C is connected to the heating element 110 to cool the heating element 110. Thus, one temperature difference generating element 130 could achieve both effects of heating the reflecting element 120 and cooling the heating element 110. However, the number of temperature difference generating element 130 is not subjected to specific restrictions in the embodiments of the present invention.

In another embodiment, the high temperature end 130H of the temperature difference generating element 130 is not necessarily connected to the reflecting element 120; or the low temperature end 130C is not necessarily connected to the heating element 110.

In the present embodiment, the heating element 110 includes a digital micromirror device (DMD) 110A, a light source 110B, a circuit board (not illustrated), an image sensing element (such as element 139) or other elements that generate heat during operation. Besides, the heating element 110 of the present embodiment is more than a heater and is provided with specific functions. However, the heating element 110 also could include a heater.

As indicated in FIG. 1, the light source 110B could be realized by such as a light emitting diode or a laser light source. The light source 110B could emit a projection light L1 (represented by bold dotted lines) projected to a to-be-scanned object 10 via a projection optical path P1. The to-be-scanned object 10 could be teeth or other to-be-scanned objects with a three-dimensional contour. The projection light L1 is reflected by the to-be-scanned object 10 to form an imaging light L2 (represented by bold dotted lines), which is then projected to the image sensing element 139 via an imaging optical path P2. In an embodiment, the image sensing element 139 could be realized by a complementary metal-oxide-semiconductor (CMOS). The projection imaging module 135 may include at least one lens for guiding the projection light L1 to the reflecting element 120. Similarly, the image optical module 137 may include at least one lens for guiding the imaging light L2 to the image sensing element 139. The image sensing element 139 could sense the imaging light L2. The processor electrically connected to the image sensing element 139 (not illustrated) could analyze the three-dimensional contour of the to-be-scanned object 10 according to the sensing signal of the image sensing element 139.

The reflecting element 120 could be realized by such as a reflecting mirror. The temperature difference generating element 130 could be realized by such as a thermoelectric cooler (TEC), but the invention is not limited thereto, and any element whose two ends or two parts have temperature difference could be used as the temperature difference generating element 130 of the present invention. Although it is not illustrated, the temperature difference generating element 130 could be electrically connected to a circuit board. The circuit board could provide a power to the temperature difference generating element 130, such that temperature difference is generated between the high temperature end 130H and the low temperature end 130C of the temperature difference generating element 130. Also, the low temperature end 130C of the temperature difference generating element 130 could be connected to at least one of the heating elements 110. As indicated in FIG. 1, the low temperature end 130C of the temperature difference generating element 130 of the present embodiment is connected to the light source 1106. However, the low temperature end 130C of the temperature difference generating element 130 could be concurrently connected to the DMD 110A and the light source 1106 or concurrently connected to the DMD 110A, the light source 1106 and the image sensing element 139.

The first heat conductive element 140 connects the reflecting element 120 to the high temperature end 130H of the temperature difference generating element 130. The heat generated by the high temperature end 130H of the temperature difference generating element 130 could be conducted to the reflecting element 120 through the first heat conductive element 140 to heat the reflecting element 120 to avoid the reflecting element 120 being atomized (atomization will affect imaging quality). To put it in greater details, the oral scanner 100 normally operates within the oral cavity, and the heat at the high temperature end 130H could heat the reflecting element 120 to a temperature close to the temperature within the oral cavity to avoid the reflecting element 120 being atomized. The first heat conductive element 140 could be realized by such as a heat pipe or a thermally conductive metal sheet. The thermally conductive metal sheet could be formed of copper, iron, aluminum, or a combination thereof.

The second heat conductive element 150 connects the heating element 110 to the low temperature end 130C of the temperature difference generating element 130. The heat generated by the heating element 110 could be conducted to the low temperature end 130C of the temperature difference generating element 130 through the second heat conductive element 150 to help the heating element 110 to dissipate the heat. The second heat conductive element 150 could be realized by such as a heat pipe or a thermally conductive metal sheet. The thermally conductive metal sheet could be formed of copper, iron, aluminum, or a combination thereof. The second heat conductive element 150 could be connected to at least one of the heating elements. As indicated in FIG. 1, the second heat conductive element 150 of the present embodiment is connected to the light source 1106. However, the second heat conductive element 150 could be concurrently connected to DMD 110A and the light source 1106 or concurrently connected to DMD 110A, the light source 1106 and the image sensing element 139.

The casing 160 has a sidewall 160w and a recess 160r, wherein the recess 160r is recessed relative to the sidewall 160w. The first heat conductive element 140 and the second heat conductive element 150 could be disposed adjacent to the sidewall 160w (with or without contact). At least a portion of the temperature difference generating element 130 is recessed in the recess 160r to avoid the temperature difference generating element 130 interfering with the projection optical path P1 and/or the imaging optical path P2. In another embodiment, as long as the temperature difference generating element 130 does not interfere with the projection optical path P1 and/or the imaging optical path P2, the recess 160r could be selectively omitted, and the temperature difference generating element 130 is not disposed with the recess 160r and is disposed adjacent to the sidewall 160w (with or without contact).

As indicated in FIG. 1, the casing 160 includes a front casing 161 and a rear casing 162. The rear casing 162 and the front casing 161 are bonded such as in a detachable manner. The rear casing 162 and the front casing 161 could be bonded by way of engagement, adhesive or screws. The reflecting element 120, the temperature difference generating element 130, the projection imaging module 135, the image optical module 137, the first heat conductive element 140 and a portion of the second heat conductive element 150 could be disposed on the front casing 161, and the heating element 110, the image sensing element 139 and another portion of the second heat conductive element 150 could be disposed on the rear casing 162. However, the arrangement of elements exemplified above depends on actual situation and is not subjected to specific restrictions in the embodiments of the present invention. In another embodiment, the temperature difference generating element 130 could be disposed on the rear casing 162.

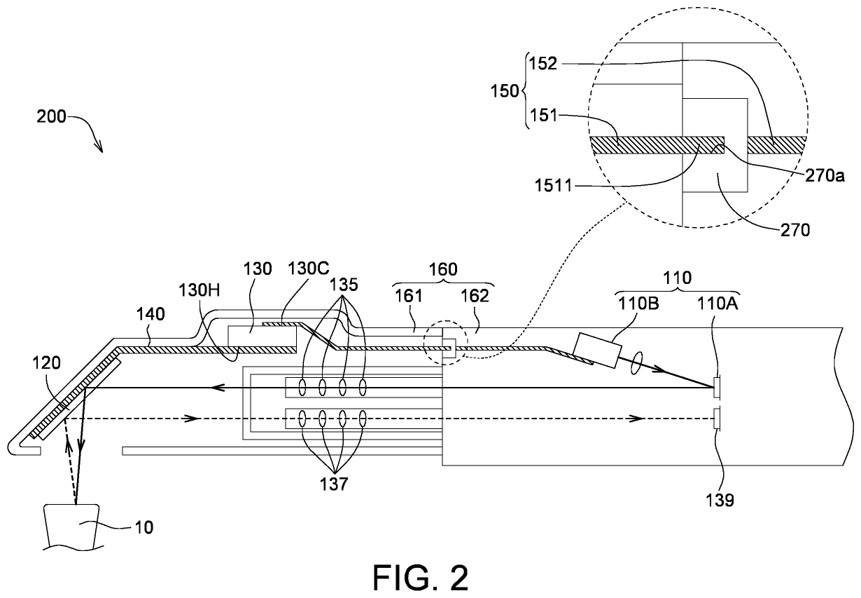

Referring to FIG. 2, a schematic diagram of an oral scanner 200 according to another embodiment of the present invention is shown. The oral scanner 200 includes a heating element 110, a reflecting element 120, a temperature difference generating element 130, a projection imaging module 135, an image optical module 137, an image sensing element 139, a first heat conductive element 140, a second heat conductive element 150, a casing 160 and an adaptor 270.

As indicated in FIG. 2, the second heat conductive element 150 includes a first section 151 and a second section 152. The first section 151 is disposed in the front casing 161 and is connected to the low temperature end 130C of the temperature difference generating element 130. The second section 152 is disposed in the rear casing 162 and is connected to the heating element 110. The first section 151 and the second section 152 are detachably bonded, such that the front casing 161 and the rear casing 162 of the casing 160 could be completely separated from each other.

As indicated in FIG. 2, the adaptor 270 could be disposed on the rear casing 162. The adaptor 270 is fixedly connected to one of the first section 151 and the second section 152 and is detachably connected to the other one of the first section 151 and the second section 152. In the present embodiment, the adaptor 270 is fixedly connected to the second section 152 and is detachably connected to the first section 151. The adaptor 270 has an engaging hole 270a, and one end 1511 of the first section 151 of the second heat conductive element 150 could be inserted into the engaging hole 270a to temporarily fix the adaptor 270.

The adaptor 270 is a transfer station between the first section 151 and the second section 152, such that the heat generated by the second section 152 could be conducted to first section 151 through the adaptor 270. The adaptor 270 could be formed of a thermal conductive material such as copper, iron, aluminum, or a combination thereof.

In another embodiment, the adaptor 270 could also be disposed on the front casing 161. In this example, the adaptor 270 is fixedly connected to the first section 151 and is detachably connected to the second section 152.

To summarize, the oral scanner disclosed in above embodiments of the present invention may include a heating element (selectively), a reflecting element (selectively) and a temperature difference generating element. The temperature difference generating element has a high temperature end and a low temperature end. The high temperature end is connected to the reflecting element to heat the reflecting element, and the low temperature end is connected to the heating element to cool the heating element. Thus, the temperature difference generating element could achieve both effects of heating the reflecting element and cooling the heating element. In another embodiment, the oral scanner could dispense with the heating element, and the temperature difference generating element still could heat the reflecting element. In other embodiments, the oral scanner could dispense with the reflecting element, and the temperature difference generating element still could cool the heating element.

While the invention has been described by way of example and in terms of the preferred embodiment (s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An oral scanner, comprising:
a heating element;
a reflecting element; and
a temperature difference generating element having a high temperature end and a low temperature end, wherein the high temperature end is connected to the reflecting element to heat the reflecting element, and the low temperature end is connected to the heating element to cool the heating element.

2. The oral scanner according to claim 1, wherein the number of the temperature difference generating element is singular.

3. The oral scanner according to claim 1, further comprising:
a first heat conductive element configured to connect the reflecting element to the high temperature end of the temperature difference generating element.

4. The oral scanner according to claim 1, further comprising:
a casing having a recess;
wherein at least one portion of the temperature difference generating element is received in the recess.

5. The oral scanner according to claim 1, further comprising:
a front casing; and
a rear casing detachably connected to the front casing;
wherein the reflecting element and the temperature difference generating element are disposed on the front casing, and the heating element is disposed on the rear casing.

6. The oral scanner according to claim 1, wherein the temperature difference generating element is a thermoelectric cooler.

7. The oral scanner according to claim 1, further comprising:
a second heat conductive element configured to connect the heating element to the low temperature end of the temperature difference generating element.

8. The oral scanner according to claim 7, wherein the second heat conductive element comprises a first section and a second section, the oral scanner further comprises:
a front casing; and
a rear casing detachably connected to the front casing;
wherein the first section is disposed in the front casing and is connected to the low temperature end of the temperature difference generating element, and the second section is disposed in the rear casing and is connected to the heating element; the first section and the second section are detachably bonded.

9. The oral scanner according to claim 8, further comprising:
an adaptor fixedly connected to one of the first section and the second section and detachably connected to the other one of the first section and the second section.

10. An oral scanner, comprising:
a heating element; and
a temperature difference generating element having a high temperature end and a low temperature end, wherein the low temperature end is connected to the heating element to cool the heating element.

11. The oral scanner according to claim 10, wherein the number of the temperature difference generating element is singular.

12. The oral scanner according to claim 10, further comprising:
a heat conductive element configured to connect the heating element to the low temperature end of the temperature difference generating element.

13. The oral scanner according to claim 10, wherein the temperature difference generating element is a thermoelectric cooler.

14. The oral scanner according to claim 10, further comprising:
a casing having a recess;
wherein at least one portion of the temperature difference generating element is received in the recess.

15. An oral scanner, comprising:
a reflecting element; and
a temperature difference generating element having a high temperature end and a low temperature end, wherein the high temperature end is connected to the reflecting element to heat the reflecting element.

16. The oral scanner according to claim 15, wherein the number of the temperature difference generating element is singular.

17. The oral scanner according to claim 15, further comprising:
a heat conductive element configured to connect the reflecting element to the high temperature end of the temperature difference generating element.

18. The oral scanner according to claim 15, wherein the temperature difference generating element is a thermoelectric cooler.

19. The oral scanner according to claim 15, further comprising:
a casing having a recess;
wherein at least one portion of the temperature difference generating element is received in the recess.
